SEQUENCE LISTING (2 sequences)

SEQ ID NO: 1 - DNA, 12 bp, Artificial
  Synthetic oligonucleotide
gcattcggactc

SEQ ID NO: 2 - DNA, 12 bp, Artificial
  Synthetic oligonucleotide.
gagtccgaatgc

(19) United States
(12) Patent Application Publication
(10) Pub. No.: US 2007/0003949 A1
(43) Pub. Date: Jan. 4, 2007

(54) Sequencing Using Tag Array

(75) Inventor: Richard P. Rava, Redwood City, CA (US)

Correspondence Address: AFFYMETRIX, INC;ATTN: CHIEF IP COUNSEL, LEGAL DEPT., 3420 CENTRAL EXPRESSWAY, SANTA CLARA, CA, 95051, US

(73) Assignee: Affymetrix, INC., Santa Clara, CA (US)

(21) Appl. No.: 11/379,992

(22) Filed: Apr. 24, 2006

Related U.S. Application Data
(60) Provisional application No. 60/673,835, filed on Apr. 22, 2005.

(51) Int. Cl. C12Q 1/68; C12P 19/34

(52) U.S. Cl. 435/6; 435/91.2

(57) ABSTRACT

The present invention provides methods for determining the sequence of a target nucleic acid with molecular inversion probes. Precircle probes are circularized if the corresponding target is present and the associated tag sequence is amplified and detected by hybridization to an array of probes that are tag complements. The presence of a tag complement indicates the presence of the corresponding target domain. Methods for using molecular inversion probes for resequencing are also disclosed.

### RELATED APPLICATIONS

This application claims the priority of U.S. Provisional Application No. 60/673,835 filed Apr. 22, 2005, the entire disclosure of which is incorporated herein by reference in its entirety.

### FIELD OF THE INVENTION

The invention is directed to novel methods of de novo sequencing and re-sequencing of nucleic acids using tag arrays and molecular inversion probes.

### BACKGROUND OF THE INVENTION

Human diseases arise from a complex interaction of DNA polymorphisms or mutations and environmental factors. Single nucleotide polymorphisms (SNPs) have been identified as potentially powerful means for genetic typing, and are predicted to supersede microsatellite repeat analysis as the standard for genetic association, linkage, and mapping studies.

The major goal in human genetics is to ascertain the relationship between DNA sequence variation and phenotypic variation. For these studies, molecular polymorphisms are indispensable for conventional meiotic mapping, fine-structure mapping and haplotype analysis. However, with the contemplated sequencing of a reference human genome and identification of all human genes, studies of complex genetic disorders are expected to be more efficient if one were to systematically search all human genes for functional variants by association and linkage disequilibrium studies. This requires the development of technology and methods for the systematic discovery of genetic variation in human DNA, primarily the single nucleotide polymorphisms (SNPS) which are the most abundant.

Several different types of polymorphism have been reported. A restriction fragment length polymorphism (RFLP) means a variation in DNA sequence that alters the length of a restriction fragment as described in Botstein et al., Am. J. Hum. Genet. 32, 314-331 (1980). The restriction fragment length polymorphism may create or delete a restriction site, thus changing the length of the restriction fragment. RFLPs have been widely used in human and animal genetic analyses (see WO 90/13668; WO90/11369; Donis-Keller, Cell 51, 319-337 (1987); Lander et al., Genetics 121, 85-99 (1989)). When a heritable trait can be linked to a particular RFLP, the presence of the RFLP in an individual can be used to predict the likelihood that the animal will also exhibit the trait.

Other polymorphisms take the form of short tandem repeats (STRS) that include tandem di-, tri- and tetra-nucleotide repeated motifs. These tandem repeats are also referred to as variable number tandem repeat (VNTR) polymorphisms. VNTRs have been used in identity and paternity analysis (U.S. Pat. No. 5,075,217; Armour et al., FEBS Lett. 307, 113-115 (1992); Hom et al., WO 91/14003; Jeffreys, EP 370,719), and in a large number of genetic mapping studies.

Other polymorphisms take the form of single nucleotide variations between individuals of the same species. Such polymorphisms are far more frequent than RFLPs, STRs and VNTRs. Some single nucleotide polymorphisms occur in protein-coding sequences, in which case, one of the polymorphic forms may give rise to the expression of a defective or other variant protein. Other single nucleotide polymorphisms occur in noncoding regions. Some of these polymorphisms may also result in defective or variant protein expression (e.g., as a result of defective splicing). Other single nucleotide polymorphisms have no phenotypic effects. Single nucleotide polymorphisms occur with greater frequency and are spaced more uniformly throughout the genome than other forms of polymorphism. The greater frequency and uniformity of single nucleotide polymorphisms means that there is a greater probability that such a polymorphism will be found in close proximity to a genetic locus of interest than would be the case for other polymorphisms. The presence of SNPs may be linked to, for example, a certain population, a disease state, or a propensity for a disease state.

Generally, polymorphisms can be associated with the susceptibility to develop a certain disease or condition. The presence of polymorphisms that cause a change in protein structure are more likely to correlate with the likelihood to develop a certain type or “Trait”. Thus, it is highly desirable to dispose of methods that allow quick and cheap genotyping of subjects. Early identification of alleles that are linked to an increased likelihood of developing a condition would allow early intervention and prevention of the development of the disease.

Thus, there is a considerable demand for high throughput methods fooorrr nucleotide sequence (e.g., SNPs) identification in regions of known sequence in order to identify alleles of polymorphic genes, e.g., SNPs. There are currently many methods available to screen polymorphisms. A typical genotyping strategy involves three basic steps. The first step consists of amplifying the target DNA, which is necessary since a human genome contains 3.times.109 base pairs of DNA and most assays lack both the sensitivity and the selectivity to accurately detect a small number of bases, in particular a single base, from a mixture this complex. As a result, most strategies currently used rely on first amplifying a region of several hundred bases including the polymorphic region to be screened using PCR. This reaction requires two unique primers for each amplified region (“amplicon”). Once the complexity has been reduced, the second step in the currently used methods consists of differentially labeling the alleles so as to be able to identify the genotype. This step involves attaching some identifiable marker (e.g. fluorescent label, mass tag, etc.) in a manner which is specific to the base being assayed. The third step in currently used methods consists of detecting the allele to determine the individuals genotypes. Detection mechanisms include fluorescent signals, the polarization of a fluorescent signal, mass spectrometry to identify mass tags, etc.

Sensitivity, i.e. detection limits, remain a significant obstacle in nucleic acid detection systems, and a variety of techniques have been developed to address this issue. Briefly, these techniques can be classified as either target amplification or signal amplification. Target amplification involves the amplification (i.e. replication) of the target sequence to be detected, resulting in a significant increase in the number of target molecules. Target amplification strategies include the polymerase chain reaction (PCR), strand displacement amplification (SDA), and nucleic acid sequence based amplification (NASBA).

Alternatively, rather than amplify the target, alternate techniques use the target as a template to replicate a signaling probe, allowing a small number of target molecules to result in a large number of signaling probes, that then can be detected. Signal amplification strategies include the ligase chain reaction (LCR), cycling probe technology (CPT), invasive cleavage techniques such as INVADER. technology, Q-Beta replicase (Q.beta.R) technology, and the use of “amplification probes” such as “branched DNA” that result in multiple label probes binding to a single target sequence.

### SUMMARY OF THE INVENTION

Methods for using molecular inversion probes for resequencing target nucleic acids and for de novo sequencing of target nucleic acids are disclosed.

In some aspects, de novo sequencing uses pools of inversion probes with known, random target sequences and known tag sequences. The probes are hybridized to the nucleic acid target to be sequenced. The 5′ and 3′ target sequences on the inversion probe are ligated together to form a circle only when they hybridize so that the ends are abutted. Circularized probes are amplified and detected by hybridization to an array of probes complementary to the tag sequences. Presence of a particular tag is indicative of the presence of the contiguous sequence complementary to the 5′ and 3′ target sequences of the inversion probe.

Each precircle probe has a 5′ and 3′ target sequence that together form a complete target domain. The complete target domain is the contiguous 5′ and 3′ target sequences end to end. Each complete target domain is associated with a unique tag sequence. The presence of that tag sequence is indicative of the presence of the complement of the associated complete target domain.

In another aspect, the inversion probes are designed to hybridize to a reference sequence leaving a single base gap. The gap corresponds to the interrogation position and is filled so that the base that is added is known or determinable. A probe may be designed to interrogate each position of a reference sequence to identify variants in the sequence.

### BRIEF DESCRIPTION OF THE DRAWINGS

FIG. 1 is a schematic of an inverions probe with target sequences at the 5′ and 3′ ends.

FIG. 2 shows the inversion probe during the inversion, amplification and detection steps.

### DETAILED DESCRIPTION OF THE INVENTION

I. General

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3rd Ed., W. H. Freeman Pub., New York, N.Y. and Berg et al. (2002) Biochemistry, 5th Ed., W. H. Freeman Pub., New York, N.Y.

The present invention can employ solid substrates, including arrays in some preferred embodiments. Methods and techniques applicable to polymer (including protein) array synthesis have been described in U.S. Ser. No. 09/536,841 (now abandoned), WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/US01/04285, which are all incorporated herein by reference in their entirety for all purposes. See also, Fodor et al., Science 251(4995), 767-73, 1991, Fodor et al., Nature 364(6437), 555-6, 1993 and Pease et al. PNAS USA 91(11), 5022-6, 1994 for methods of synthesizing and using microarrays.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GENECHIP. Example arrays are shown on the website at affymetrix.com. The present invention also contemplates many uses for polymers attached to solid substrates. These uses include gene expression monitoring, profiling, library screening, genotyping and diagnostics. Gene expression monitoring and profiling methods are shown in U.S. Pat. Nos. 5,800,992, 6,013,449, 6,020,135, 6,033,860, 6,040,138, 6,177,248 and 6,309,822. Genotyping and uses therefore are shown in U.S. Ser. Nos. 60/319,253, 10/013,598, and U.S. Pat. Nos. 5,856,092, 6,300,063, 5,858,659, 6,284,460, 6,361,947, 6,368,799 and 6,333,179. Additional methods of genotyping, complexity reduction and nucleic acid amplification are disclosed in U.S. patent application Ser. Nos. 60/508,418 (now inactive), 10/912,445, 10/841,027, 10/442,021, 10/646,674, 10/712,616,U.S. Publications US20030096235, US20030232348, US20040132056, US20040110153, US20040146890 and U.S. Pat. No. 6,582,938. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, e.g., PCR Technology: Principles and Applications for DNA Amplification (Ed. H. A. Erlich, Freeman Press, N.Y., N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188,and 5,333,675, and each of which is incorporated herein by reference in their entireties for all purposes. In addition, there are a number of variations of PCR which also find use in the invention, including “quantitative competitive PCR” or “QC-PCR”, “arbitrarily primed PCR” or “AP-PCR”, “immuno-PCR”, “Alu-PCR”, “PCR single strand conformational polymorphism” or “PCR-SSCP”, allelic PCR (see Newton et al. Nucl. Acid Res. 17:2503 91989); “reverse transcriptase PCR” or “RT-PCR”, “biotin capture PCR”, “vectorette PCR”, “panhandle PCR”, and “PCR select cDNA subtraction”, for example. The sample may be amplified on the array. See, for example, U.S. Pat. No. 6,300,070 and U.S. Ser. No. 09/513,300 (now abandoned), which are incorporated herein by reference.

Other suitable amplification methods include the ligase chain reaction (LCR) (e.g., Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self sustained sequence replication (Guatelli et al., Proc. Nat. Acad. Sci. USA, 87, 1874 (1990) and WO90/06995), selective amplification of target polynucleotide sequences (U.S. Pat. No 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA). (See, U.S. Pat. Nos. 5,409,818, 5,554,517, and 6,063,603, each of which is incorporated herein by reference). Strand displacement amplification (SDA) is generally described in Walker et al., in Molecular Methods for Virus Detection, Academic Press, Inc., 1995, and U.S. Pat. Nos. 5,455,166 and 5,130,238, all of which are hereby incorporated by reference. Other amplification methods that may be used are described in, U.S. Pat. Nos. 6,582,938, 5,242,794, 5,494,810, 4,988,617.

Cycling probe technology (CPT) is a nucleic acid detection system based on signal or probe amplification rather than target amplification, such as is done in polymerase chain reactions (PCR). Cycling probe technology relies on a molar excess of labeled probe which contains a scissile linkage of RNA. Upon hybridization of the probe to the target, the resulting hybrid contains a portion of RNA:DNA. This area of RNA:DNA duplex is recognized by RNAseH and the RNA is excised, resulting in cleavage of the probe. The probe now consists of two smaller sequences which may be released, thus leaving the target intact for repeated rounds of the reaction. The unreacted probe is removed and the label is then detected. CPT is generally described in U.S. Pat. Nos. 5,011,769, 5,403,711, 5,660,988, and 4,876,187, and PCT published applications WO 95/05480, WO 95/1416, and WO 95/00667, all of which are specifically incorporated herein by reference.

Additional methods of sample preparation and techniques for reducing the complexity of a nucleic sample are described in Dong et al., Genome Research 11, 1418 (2001), in U.S. Pat. Nos. 6,632,611, 6,361,947, 6,391,592 and U.S. patent application Ser. No. 09/916,135, US publications US20030036069 and US20030096235.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2nd Ed. Cold Spring Harbor, N.Y., 1989); Berger and Kimmel Methods in Enzymology, Vol. 152, Guide to Molecular Cloning Techniques (Academic Press, Inc., San Diego, Calif., 1987); Young and Davis, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Patent Application 20040012676 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Publication US20040012676 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001). See U.S. Pat. No. 6,420,108.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. Publications No. US20020183936 and US20040049354.

II. Definitions

The term “array” as used herein refers to an intentionally created collection of molecules which can be prepared either synthetically or biosynthetically. The molecules in the array can be identical or different from each other. The array can assume a variety of formats, for example, libraries of soluble molecules; libraries of compounds tethered to resin beads, silica chips, or other solid supports.

Preferred arrays typically comprise a plurality of different nucleic acid probes that are coupled to a surface of one or more substrates in different, known or determinable locations. Arrays have been generally described in, for example, U.S. Pat. Nos. 5,143,854, 5,445,934, 5,744,305, 5,677,195, 5,800,992, 6,040,193, 5,424,186 and Fodor et al., Science, 251:767-777 (1991).

Arrays may generally be produced using a variety of techniques, such as mechanical synthesis methods or light directed synthesis methods that incorporate a combination of photolithographic methods and solid phase synthesis methods. Techniques for the synthesis of these arrays using mechanical synthesis methods are described in, e.g., U.S. Pat. Nos. 5,384,261 and 6,040,193. Arrays may be nucleic acids on beads, gels, polymeric surfaces, fibers such as fiber optics, glass or any other appropriate substrate. (See U.S. Pat. Nos. 5,770,358, 5,789,162, 5,708,153, 6,040,193 and 5,800,992.)

Arrays may be packaged in such a manner as to allow for diagnostic use or can be an all-inclusive device; e.g., U.S. Pat. Nos. 5,856,174 and 5,922,591. Preferred arrays are commercially available from Affymetrix (Santa Clara, Calif.) under the brand name GeneChip® and are directed to a variety of purposes, including genotyping and gene expression monitoring for a variety of eukaryotic and prokaryotic species.

The term “combinatorial synthesis strategy” as used herein refers to a combinatorial synthesis strategy is an ordered strategy for parallel synthesis of diverse polymer sequences by sequential addition of reagents which may be represented by a reactant matrix and a switch matrix, the product of which is a product matrix. A reactant matrix is a l column by m row matrix of the building blocks to be added. The switch matrix is all or a subset of the binary numbers, preferably ordered, between l and m arranged in columns. A “binary strategy” is one in which at least two successive steps illuminate a portion, often half, of a region of interest on the substrate. In a binary synthesis strategy, all possible compounds which can be formed from an ordered set of reactants are formed. In most preferred embodiments, binary synthesis refers to a synthesis strategy which also factors a previous addition step. For example, a strategy in which a switch matrix for a masking strategy halves regions that were previously illuminated, illuminating about half of the previously illuminated region and protecting the remaining half (while also protecting about half of previously protected regions and illuminating about half of previously protected regions). It will be recognized that binary rounds may be interspersed with non-binary rounds and that only a portion of a substrate may be subjected to a binary scheme. A combinatorial “masking” strategy is a synthesis which uses light or other spatially selective deprotecting or activating agents to remove protecting groups from materials for addition of other materials such as amino acids.

The term “complementary” as used herein refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984).

The term “genome” as used herein is all the genetic material in the chromosomes of an organism. DNA derived from the genetic material in the chromosomes of a particular organism is genomic DNA. A genomic library is a collection of clones made from a set of randomly generated overlapping DNA fragments representing the entire genome of an organism.

The term “isolated nucleic acid” as used herein mean an object species invention that is the predominant species present (i.e., on a molar basis it is more abundant than any other individual species in the composition). Preferably, an isolated nucleic acid comprises at least about 50, 80 or 90% (on a molar basis) of all macromolecular species present. Most preferably, the object species is purified to essential homogeneity (contaminant species cannot be detected in the composition by conventional detection methods).

The phrase “massively parallel screening” refers to the simultaneous screening of from about 100, 1000, 10,000 or 100,000 to 1000, 10,000, 100,000, 1,000,000 or 3,000,000 or more different nucleic acid hybridizations.

The term “microtiter plates” as used herein refers to arrays of discrete wells that come in standard formats (96, 384 and 1536 wells) which are used for examination of the physical, chemical or biological characteristics of a quantity of samples in parallel.

The term “mixed population” or sometimes refer by “complex population” as used herein refers to any sample containing both desired and undesired nucleic acids. As a non-limiting example, a complex population of nucleic acids may be total genomic DNA, total genomic RNA or a combination thereof. Moreover, a complex population of nucleic acids may have been enriched for a given population but include other undesirable populations. For example, a complex population of nucleic acids may be a sample which has been enriched for desired messenger RNA (mRNA) sequences but still includes some undesired ribosomal RNA sequences (rRNA).

The term “nucleic acids” as used herein may include any polymer or oligomer of pyrimidine and purine bases, preferably cytosine, thymine, and uracil, and adenine and guanine, respectively. See Albert L. Lehninger, PRINCIPLES OF BIOCHEMISTRY, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

The term “oligonucleotide” or sometimes refer by “polynucleotide” as used herein refers to a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA) which may be isolated from natural sources, recombinantly produced or artificially synthesized and mimetics thereof. A further example of a polynucleotide of the present invention may be peptide nucleic acid (PNA). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

A nucleic acid of the present invention will generally contain phosphodiester bonds, although in some cases, such as in the design of probes, nucleic acid analogs are included that may have alternate backbones, comprising, for example, phosphoramide (Beaucage et al., Tetrahedron 49(10):1925 (1993) and references therein; Letsinger, J. Org. Chem. 35:3800 (1970): Sprinzl et al., Eur. J. Biochem. 81:579 (1977); Letsinger et al., Nucl. Acids Res. 14:3487 (1986); Sawai et al., Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Mejer et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positively charged backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1998)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp 169-176). Several nucleic acid analogs are described in Rawls, C & E News Jun. 2, 1997 page 35. These modifications of the ribose-phosphate backbone may be done to facilitate the addition of labels, or to increase the stability and half-life of such molecules in physiological environments.

Pharmacogenomics is the study of the relationship between an individual's genotype and that individual's response to a foreign compound or drug. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining the type of drug and dosage and/or therapeutic regimen of treatment.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(1-11):983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofarans) and consumption of fava beans. Thus, it would be highly desirable to dispose of fast and cheap methods for determining a subject's genotype so as to predict the best treatment.

The term “primer” as used herein refers to a single-stranded oligonucleotide capable of acting as a point of initiation for template-directed DNA synthesis under suitable conditions for example, buffer and temperature, in the presence of four different nucleoside triphosphates and an agent for polymerization, such as, for example, DNA or RNA polymerase or reverse transcriptase. The length of the primer, in any given case, depends on, for example, the intended use of the primer, and generally ranges from 15 to 30 nucleotides. Short primer molecules generally require cooler temperatures to form sufficiently stable hybrid complexes with the template. A primer need not reflect the exact sequence of the template but must be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

The term “probe” as used herein refers to a surface-immobilized molecule that can be recognized by a particular target. See U.S. Pat. No. 6,582,908 for an example of arrays having all possible combinations of probes with 10, 12, and more bases. Examples of probes that can be investigated by this invention include, but are not restricted to, agonists and antagonists for cell membrane receptors, toxins and venoms, viral epitopes, hormones (for example, opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

The term “tag” or “tag sequence” is a selected nucleic acid with a specified nucleic acid sequence. A tag probe has a region that is complementary to a selected tag. A set of tags or a collection of tags is a collection of specified nucleic acids that may be of similar length and similar hybridization properties, for example similar Tm. The tags in a collection of tags bind to tag probes with minimal cross hybridization so that a single species of tag in the tag set accounts for the majority of tags which bind to a given tag probe species under hybridization conditions. For additional description of tags and tag probes and methods of selecting tags and tag probes see U.S. Pat. No. 6,458,530 and EP/0799897, each of which is incorporated herein by reference in their entirety.

The term “target sequence”, “target nucleic acid” or “target” refers to a nucleic acid of interest. The target sequence may or may not be of biological significance. Typically, though not always, it is the significance of the target sequence which is being studied in a particular experiment. As non-limiting examples, target sequences may include regions of genomic DNA which are believed to contain one or more polymorphic sites, DNA encoding or believed to encode genes or portions of genes of known or unknown function, DNA encoding or believed to encode proteins or portions of proteins of known or unknown function, DNA encoding or believed to encode regulatory regions such as promoter sequences, splicing signals, polyadenylation signals, etc.

Target sequences may be interrogated by hybridization to an array. The array may be specially designed to interrogate one or more selected target sequence. The array may contain a collection of probes that are designed to hybridize to a region of the target sequence or its complement. Different probe sequences are located at spatially addressable locations on the array. For genotyping a single polymorphic site probes that match the sequence of each allele may be included. At least one perfect match probe, which is exactly complementary to the polymorphic base and to a region surrounding the polymorphic base, may be included for each allele. In a preferred embodiment the array comprises probes that include 12 bases on either side of the SNP. Multiple perfect match probes may be included as well as mismatch probes.

Hybridization probes are oligonucleotides capable of binding in a base-specific manner to a complementary strand of nucleic acid. Such probes include peptide nucleic acids, as described in Nielsen et al., Science 254, 1497-1500 (1991), and other nucleic acid analogs and nucleic acid mimetics. See U.S. Pat. No. 6,156,501.

III. Sequencing with Molecular Inversion Probes

Genomic variation between individuals is believed to account for more than 90% of all differences between individuals. This variation is typically found in the form of polymorphisms with single nucleotide polymorphisms (SNPs) accounting for the majority of genetic variation. Understanding the relationship between genetic variation and biological function on a genomic scale is expected to provide insight into the biology of humans and other species, including those that cause disease in humans. Identification of large numbers of SNPs will be integral to furthering our understanding. In addition to the common sequence variants that are found in populations, typically at a frequency of at least 1% in a population, there are also more rare variants, typically occurring at a frequency of less than 1% in a population. The common variants are often referred to as polymorphisms or SNPs whereas the more rare variants are often referred to as “mutations”. Sequence variants may be neutral and have no detectable impact on phenotype or they may result in or contribute to a particular phenotype, for example, they may cause or contribute to disease. The term “mutation” is sometimes associated with disease causing change, whereas the majority of polymorphisms are probably not associated with disease. Methods for detecting the presence of rare or previously uncharacterized variants are also provided by the present disclosure.

Arrays of probes provide an efficient means of analyzing variant sequences. Array-based resequencing has been used, for example, in the identification of large numbers of human polymorphisms in mitochondrial DNA and ESTs, the identification of drug-induced mutations in HIV, and analysis of mutations in p53 correlated with human cancer.

In one embodiment, the method is directed to detect polymorphisms in a selected sequence or sequences by re-sequencing the sequence(s) from a plurality of individuals or sources.

The selected sequence may first be identified by using a genotyping method that identifies a large region of interest, for example a linkage analysis study or an association study. Sequence(s) of interest may be downloaded from any number of public or proprietary databases. The polymorphisms may be novel polymorphisms or polymorphisms that are known to occur in one or more population.

Molecular Inversion Probe (MIP) technology has been described in U.S. Pat. Nos. 6,858,412, 5,866,337 and 5,871,921, which are incorporated herein by reference. In particular, MIP technology has been shown to be an efficient and scallable method to perform multiplex SNP assays.

The present invention is directed to novel methods of multiplexing amplification, particularly polymerase chain reaction (PCR) reactions, to detect the presence of selected sequences. PCR is a preferred method of amplification, although as described herein a variety of amplification techniques can be used. As will be appreciated by those in the art, there are a wide variety of configurations and assays that can be used. There are two general methodologies: a “one step” and a “two step” process.

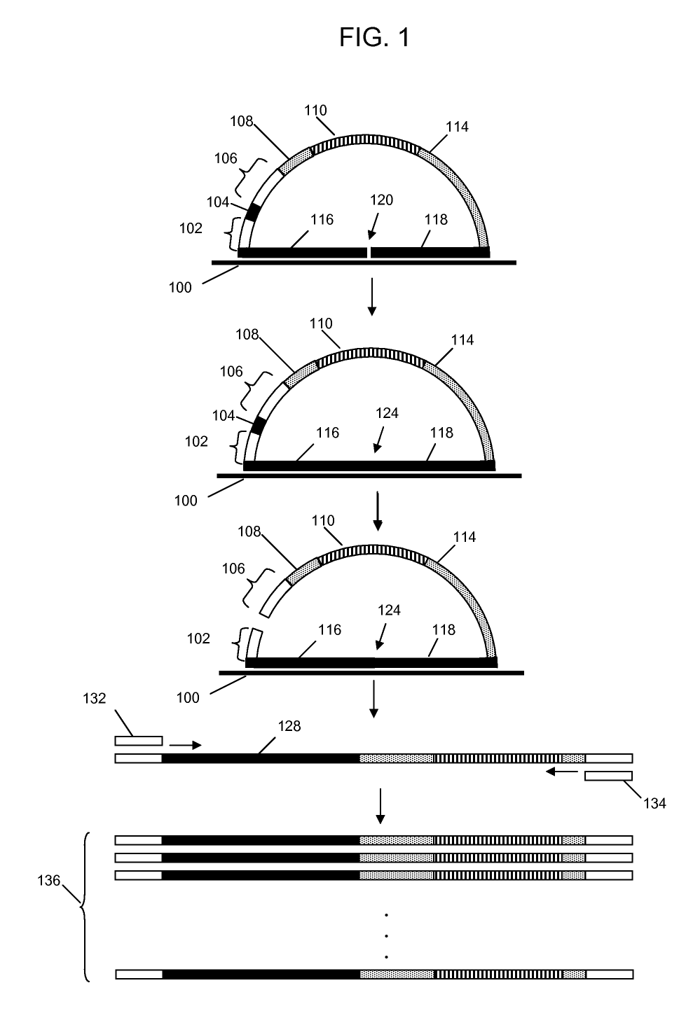

The “one step” process can generally be described as follows. A collection of precircle probes is added to a target sequence from a sample that contains a nucleic acid to be sequenced. The collection of precircle probes contains a plurality of different probe sequences. As shown in FIG. 1, each precircle probe has a first target domain 116 and a second target domain 118 that are of known sequence. Each probe also has a barcode or tag sequence 110 that is also known. Each different barcode sequence is associated with a different sequence at 116 and 118. When a precircle probe is hybridized to a target sequence so that the ends of the probe are immediately adjacent to one another there is a separation 120 between the ends that can be closed by ligation to form a phosphate bond between the ends at 124. The sample can be treated with exonuclease to selectively remove precircle probes that have not formed closed circles. The closed circle probes can be cleaved at 104 to form open circles flanked at the 5′ and 3′ ends by priming sites 102 and 106. The probe may be amplified using primers 132 and 134 to priming sites 102 and 106. The amplification products 136 can be analyzed by hybridization to an array of probes that are complementary to the barcode sequences 110. The presence of a particular barcode sequence in the amplification product 136 is indicative of the presence of the sequence 128 that is formed by ligation of 116 to 118.

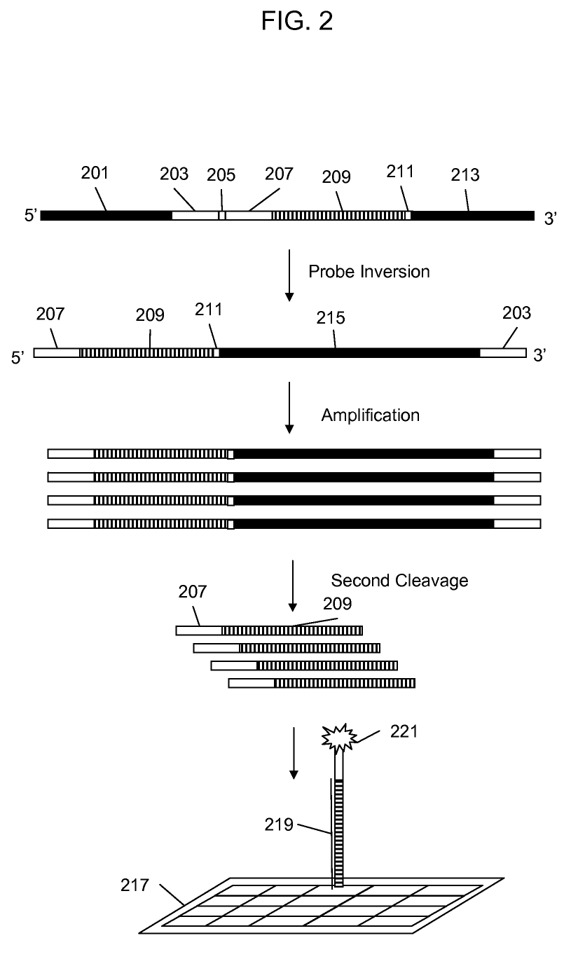

The process of probe inversion, amplification and detection is further illustrated in FIG. 2. The probe sequence has a 5′ end target domain 201 and a 3′ end target domains 213, tag 209, universal priming sequences 203 and 207, first cleavage site 205 and second cleavage site 211. After circularization by joining the 5′ and 3′ ends and cleavage at 205 an inverted probe is generated. The inverted probe has priming sequence 207 at the 5′ end and priming sequence 203 at the 3′ end. Target complementary regions 201 and 213 are now end to end to form 215. The inverted probe can be amplified using primers to 207 and 203 and the amplification product may be cleaved at 211. In some aspects 211 is a restriction site and the amplification product is double stranded. Cleavage at 211 generates fragments with 207 and 209 separated from the rest of the amplified probe. This fragment can be detected by hybridization to an array 217 of probes complementary to the tag sequences. For example, tag probe 219 is perfectly complementary to tag 209. The cleavage product is shown hybridized to the array with detectable label 221 attached to 207.

As outlined more fully below, these target domains in the target sequence are directly adjacent to one another in a preferred embodiment, although in some aspects they can be separated by a gap of one or more nucleotides. The precircle probe comprises first and second targeting domains at its termini that are substantially complementary to the target domains of the target sequence. The precircle probe comprises one or optionally more universal priming sites, separated by a cleavage site, and a barcode sequence. If there is no gap between the target domains of the target sequence, and the 5′ and 3′ nucleotides of the precircle probe are perfectly complementary to the corresponding bases at the junction of the target domains, then the 5′ and 3′ nucleotides of the precircle probe are “abutting” each other and can be ligated together, using a ligase, to form a closed circular probe. The 5′ and 3′ end of a nucleic acid molecule are referred to as “abutting” each other when they are in contact close enough to allow the formation of a covalent bond, in the presence of ligase and adequate conditions.

This method is based on the fact that the two targeting domains of a precircle probe can be preferentially ligated together, if they are hybridized to a target strand such that they abut and if perfect complementarity exists at the two bases being ligated together. Perfect complementarity at the termini allows the formation of a ligation substrate such that the two termini can be ligated together to form a closed circular probe. If this complementarity does not exist, no ligation substrate is formed and the probes are not ligated together to an appreciable degree.

Once the precircle probes have been ligated, the unligated precircle probes and/or target sequences are optionally removed or inactivated. The closed circular probe is then linearized by cleavage at the cleavage site, resulting in a cleaved probe comprising the universal priming sites at the new termini of the cleaved probe. The addition of universal primers, an extension enzyme such as a polymerase, and NTPs results in amplification of the cleaved probe to form amplicons. These amplicons can be detected in a variety of ways. For example, in the case where barcode sequences are used, the amplicons containing the barcodes can then be added to universal biochip arrays, as is well known in the art although as will be appreciated by those in the art, a number of other detection methods, including solution phase assays, can be run.

In addition to the targeting domains and universal priming sites, the precircle probes preferably comprise at least a first cleavage site. Preferred cleavage sites are those that allow cleavage of nucleic acids in specific locations. Suitable cleavage sites include, but are not limited to, the incorporation of uracil or other ribose nucleotides, restriction endonuclease sites, etc.

In a preferred embodiment, the cleavage site comprises a uracil base. This allows the use of uracil-N-glycolylase, an enzyme which removes the uracil base while leaving the ribose intact. This treatment, combined with changing the pH (to alkaline) by heating, or contacting the site with an apurinic endonuclease that cleaves basic nucleosides, allows a highly specific cleavage of the closed circle probe.

In one embodiment, a restriction endonuclease site is used, preferably a rare one. As will be appreciated by those in the art, this may require the addition of a second strand of nucleic acid to hybridize to the restriction site, as many restriction endonucleases require double stranded nucleic acids upon which to work. In one embodiment, the restriction site can be part of the primer sequence such that annealing the primer will make the restriction site double-stranded and allow cleavage.

In a preferred embodiment, there is a gap between the target domains of the target sequence. In the case of a genotyping reaction, there is a single nucleotide gap, comprising the detection position, e.g. the SNP position. The addition of a single type of dNTP and a polymerase to the hybridization complex to “fill” the gap, if the dNTP is perfectly complementary to the detection position base. The dNTPs are optionally removed, and the ligase is added to form a closed circle probe. The cleavage, amplification and detection proceeds as above.

Alternatively, there may be a gap of more than one nucleotide between the target domains. In this case, as is more fully outlined below, either a plurality of dNTPs, a “gap oligonucleotide” as generally depicted in FIG. 3C or a precircle probe with a “flap” as is generally depicted in FIG. 3D can be used to accomplish the reaction.

The “two step” process is similar to the process outlined above. However, in this embodiment; after the precircle probe has been circularized, a single universal primer is added, in the presence of a polymerase and dNTPs, such that a new linear copy of the closed probe is produced, with new termini. This linearized closed probe is then amplified as more fully described below. The “two-step” process is particularly advantageous for reducing unwanted background signals arising from subsequent amplification reactions. This can be achieved by designing the cleavage sites into the precircle probes that when cleaved will prevent any amplification of any probe. Additional background reduction processes may also be incorporated into the compositions and methods of the present invention and are discussed in more detail herein.

The methods of the invention are particularly advantageous in reducing problems associated with cross-hybridizations and interactions between multiple probes, which can lead to unwanted background amplification. By circularizing the precircle probes and treating the reaction with exonuclease, linear nucleic acids are degraded and thus cannot participate in amplification reactions. This allows the methods of the invention to be more robust and multiplexable than other amplification methods that rely on linear probes.

Accordingly, the present invention provides compositions and methods for detecting, quantifying and/or genotyping target nucleic acid sequences in a sample. In general, the genotyping methods described herein relate to the detection of nucleotide substitutions, although as will be appreciated by those in the art, deletions, insertions, inversions, etc. may also be detected.

In a preferred embodiment, a method for re-sequencing or de-novo (without prior knowledge) sequencing a target nucleic acid using random molecular inversion probes and tag arrays is provided. In a preferred embodiment, the target nucleic acid is genomic DNA. Random molecular inversion probes with random sequences at both ends are hybridize to the target nucleic acid at regions where complementarities exists between the given random sequences and the target sequence. Random sequences are preferably about 6 nucleotides long (random hexamers) but may be longer, for example, 9 to 15 nucleotides long). In a preferred embodiment, a probe comprises random sequences at its 5′ and 3′ end, a tag sequence, at least one universal priming site and a cleavage site. Sequence-tagged molecular inversion probes have been described, e.g. in Hardenbol P. (2003), Nat. Biotech., 21(6) pp 673-678, Hardenbol et al., Genome Res 15:269-75 (2005) and U.S. Patent Application 20040101835, all of which are incorporated by reference.

In one embodiment, the precircle probes have random end sequences that when hybridized to a target so that the ends are directly adjacent or abutting so that there is no gap between the two domains of the target sequence. The 5′ and 3′ nucleotides of the probe can be ligated together using a ligase to form a closed circular probe. If the ends are separated by a gap of one or more nucleotides the gap may be filled. In a preferred embodiment the gap is filled by a sequence of known length, for example, by addition of a single base to the 3′ end of the precircle probe that may correspond to a polymorphisms or by ligation of a gap filling oligonucleotide of known length into the gap.

Once the ends of the probes have been ligated to form closed circle probes, the unligated probes and/or the target sequences are optionally removed or inactivated. For example, exonucleases are added that will degrade any linear nucleic acids, leaving the closed circular probes. The closed circular probes may then be linearized by cleavage at the cleavage site resulting in linear cleaved probes comprising a universal priming site at the new termini of the probes. In one embodiment, the cleavage site comprises a uracil base. This allows the use of uracil-N-glycosylase, an enzyme that removes the uracil base wile leaving the ribose intact. This treatment, combined with changing the pH to alkaline by heating allows a highly specific cleavage of the closed circle probe. In one embodiment, a restriction endonuclease site is used, preferable a rare one. In one embodiment, the addition of universal primers, an extension enzyme such as a polymerase and dNTPs results in amplification of the linear probes. In a preferred embodiment, the amplified probes comprise a detectable label. A wide variety of labels suitable for labeling nucleic acids are known and reported extensively in both the scientific and patent literature, and are generally applicable to the present invention for the labeling of tag probes and amplified tag probes for detection by oligonucleotides arrays. In one embodiment, the barcode sequences are 19 to 25 mers that are selected from all possible 19 to 25 mers to have similar hybridization characteristics such as melting temperature and minimal homology to sequences in the public database. In a preferred embodiment, the barcode sequences associated with the random end pairs are detected using an oligonucleotide Tag-array such as the GENFLEX Tag Array (Affymetrix Inc., CA) or the Universal Tag Arrays available as 3 k, 5K, 10K and 35K. “Tag” or “barcode” sequence refers to the sequence that is being captured by the array. This is represented as 110 in FIG. 1 and 209 in FIG. 2. As shown in FIG. 2 the amplification product may be cleaved to release the primer sequence 207 and the barcode sequence 209 from the remainder of the amplified probe. The tag sequence 209 is detected by hybridization to an array 211 of probes 213 that are perfectly complementary to the tag sequence 209. The probes or tag complements are attached to the solid support. Each feature of the array has probes that are complementary to a different tag sequence.

The tag-arrays can have any number of different oligonucleotide sets, determined by the number of nucleic acid tags to be screened against the array in a given application. In one embodiment, the array has from 3000 to 10,000, or 10,000 to 100,000, 100,000 to 1,000,000, 1,000,000 to 10,000,000 or 10,000 to 50,000,000 different features with each feature containing a different tag probe. See U.S. Pat. No. 6,458,530 and EP0799897, each of which is incorporated herein by reference in their entirety.

The methods may be used to perform de novo resequencing or re-sequencing using a reference sequence. In preferred aspects each precircle probe contains a first sequence at 201 and a second sequence at 213 that when combined make a contiguous sequence 215. The sequence 215 is 5′ to 3′ the same as 213 plus 201. So, for example, if 213 is 5′-GCATTC-3′ and 201 is 5′-GGACTC-3′ then 215 is 5′-GCATTCGGACTC-3′ (SEQ ID NO. 1). If tag 209 is detected on the array this indicates that the sequence 5′-GAGTCCGAATGC-3′ (SEQ ID NO. 2) that is the complement of SEQ ID NO 1 was present in the sample.

In a preferred aspect the sequences present at 213 and 201, which combine to make sequence 215, represent all possible non-complementary 12 mers. There are 4¹² possible 12 mers or 16,777,216. Removing complements there are 8,388,608 different non-complementary 12 mers. Complements may be removed if the target to be sequenced is double stranded. In one aspect there are about 8 million different precircle probes generated. Each precircle probe has a different 12 mer (6 bases at the 3′ target end and 6 bases at the 5′ target end) and a different tag sequence. The tags may be 15 to 25 bases and 12 mers that are complementary to the tags are not included in the set. The tag sequences may be selected to have a common sequence feature that is eliminated from the 12 mer probe sequences, for example, if each 20 mer tag has CCCC in the center of the tag and none of the 12 mers have GGGG then none of the 12 mers should be perfectly complementary to any of the tags. Similarly, 12 mers that could be circularized using the common sequences of the precircle probes, for example, complementary to the priming sites, are also removed from the set of 12 mers. This design prevents the precircle probes from hybridizing to the tag or common sequences of another precircle probe and circularizing in the absence of the complementary target in the nucleic acid being sequenced. In one aspect tags are separated from complementary there are at least two pools of precircle probes and the precircle probes that have target sequences that are complementary to tag sequences are separated from precircle probes with those tag sequences and are processed in separate reactions.

The methods may also be used for resequencing a target. The sequences present at the target domains are designed to be complementary to the known reference sequences flanking an interrogation position with a gap for the interrogation position. In a preferred aspect the gap is a single base corresponding to the interrogation position. The free 3′ end of the target domain is extended by a single base that is complementary to and indicative of the interrogation position. The base is added in a manner that allows for determination of the identity of the added base. For example, all four dNTPs may be present but each may be labeled with a different label. In another aspect there may be four separate reactions each with a different dNTP present.

Target for resequencing may be prepared, for example, by target specific long range PCR in either single or multiplex. This may be used to reduce the complexity of the sequence to be resequenced. Resequencing by hybridization and methods for preparing samples for resequencing have been disclosed, for example, in Cutler et al., Genome Res., 11:1913-25 (2001), Maitra et al., Genome Res 14:812-9 (2004) and Tsolaki et al., RNAS 101:4865-70 (2004).

In some aspects, the nucleic acid sample to be analyzed by de novo sequencing using the disclosed methods is a reduced complexity sample, for example, a sample that has been enriched for a portion of a genome, for example, a 5, 10, 20, 100, 300 or 500 kilobase amplification product. In another aspect a bacterial or viral genome may be interrogated. Repetitive sequence may be removed prior to analysis.

In a preferred embodiment, the products are labeled with a detection label. In some aspects the detection label can be directly detected, by spectroscopic, photochemical, biochemical, immunochemical, electrical, optical or chemical means. Useful labels in the present invention include for example, fluorescein isothiocyanate, Texas red, rhodamine, dixogenin, biotin, and the like, radiolabels (e.g., 3H, 125I, 35S, 14C, 32P, 33P, etc.), enzymes (e.g., horse-radish peroxidase, alkaline phosphatase etc.) spectral calorimetric labels such as colloidal gold or colored glass or plastic (e.g. polystyrene, polypropylene, latex, etc.) beads; magnetic, electrical, thermal labels; and mass tags. Preferred labels include chromophores or phosphors but are preferably fluorescent dyes.

In another embodiment, a secondary detectable label is used. A secondary label is one that is indirectly detected; for example, a secondary label can bind or react with a primary label for detection, can act on an additional product to generate a primary label (e.g. enzymes), or may allow the separation of the compound comprising the secondary label from unlabeled materials, etc. Secondary labels include, but are not limited to, one of a binding partner pair; chemically modifiable moieties; nuclease inhibitors, enzymes such as horseradish peroxidase, alkaline phosphatases, luciferases, etc.

In a preferred embodiment, the secondary label is a binding partner pair. For example, the label may be a hapten or antigen, which will bind its binding partner. In a preferred embodiment, the binding partner can be attached to a solid support to allow separation of extended and nonextended primers. For example, suitable binding partner pairs include, but are not limited to: antigens (such as proteins (including peptides)) and antibodies (including fragments thereof (FAbs, etc.)); proteins and small molecules, including biotin/streptavidin; enzymes and substrates or inhibitors; other protein-protein interacting pairs; receptor-ligands; and carbohydrates and their binding partners. Nucleic acid-nucleic acid binding proteins pairs are also useful. In general, the smaller of the pair is attached to the NTP for incorporation into the primer. Preferred binding partner pairs include, but are not limited to, biotin (or imino-biotin) and streptavidin, dixogenin and Abs, and PROLINX reagents.

In a preferred embodiment, the binding partner pair comprises a primary detection label (for example, attached to the NTP and therefore to the amplicon) and an antibody that will specifically bind to the primary detection label. By “specifically bind” herein is meant that the partners bind with specificity sufficient to differentiate between the pair and other components or contaminants of the system. The binding should be sufficient to remain bound under the conditions of the assay, including wash steps to remove non-specific binding. In some embodiments, the dissociation constants of the pair will be less than about 10⁻⁴-10⁻⁶ M⁻¹, with less than about 10.sup.⁻⁵ to 10⁻⁹ M⁻¹ being preferred and less than about 10⁻⁷-10⁻⁹ M⁻¹ being particularly preferred.

In a preferred embodiment, the secondary label is a chemically modifiable moiety. In this embodiment, labels comprising reactive functional groups are incorporated into the nucleic acid. The functional group can then be subsequently labeled with a primary label. Suitable functional groups include, but are not limited to, amino groups, carboxy groups, maleimide groups, oxo groups and thiol groups, with amino groups and thiol groups being particularly preferred. For example, primary labels containing amino groups can be attached to secondary labels comprising amino groups, for example using linkers as are known in the art; for example, homo-or hetero-bifunctional linkers as are well known (see 1994 Pierce Chemical Company catalog, technical section on cross-linkers, pages 155-200, incorporated herein by reference).

Methods for sequencing by hybridization have been disclosed for example, in Drmanac et al., Adv Biochem Eng Biotechnol. 77:75-101 (2002), Schirinzi et al., Genet Test 10:8-17 (2006), and in U.S. Pat. Nos. 5,202,231, 5,492,806 and 5,525,464. Analysis methods for determining a sequence using hybridization are disclosed, for example, in U.S. Pat. No. 5,972,619.

### CONCLUSION

It is to be understood that the above description is intended to be illustrative and not restrictive. Many variations of the invention will be apparent to those of skill in the art upon reviewing the above description. The scope of the invention should be determined with reference to the appended claims, along with the full scope of equivalents to which such claims are entitled. All cited references, including patent and non-patent literature, are incorporated herewith by reference in their entireties for all purposes.

What is claimed is:

1. A method for detecting the presence of a target sequence in a nucleic acid sample, said method comprising:
a. mixing the nucleic acid sample with a plurality of precircle probes under conditions that allow hybridization of precircle probes to complementary target sequences in the nucleic acid sample to form a plurality of hybridization complexes, wherein each precircle probe comprises in the following order:
i. a 5′ target domain comprising a first known sequence;
ii. a first universal priming site;
iii. a first cleavage site;
iv. a second universal priming site;
v. a tag sequence;
vi. an optional second cleavage site; and
vii. a 3′ target domain comprising a second known sequence, wherein said target sequence consists of the complement of said first and second known sequences;
b. ligating together the ends of precircle probes that are hybridized to target sequences so that the 5′ and 3′ ends of the precircle probe are immediately adjacent to form closed circular probes from a plurality of said precircle probes;
c. optionally digesting any linear probes remaining after step (b);
d. cleaving said closed circular probes at the first cleavage site to obtain linear tag probes;
e. amplifying said linear tag probes to obtain amplified linear tag probes;
f. hybridizing said amplified linear tag probes to an oligonucleotide array comprising probes that are tag complements to obtain a hybridization pattern; and
g. analyzing said hybridization pattern to identify at least one tag sequence that is present in the amplified linear tag probes, wherein the presence of a selected tag sequence is indicative of the presence of the target sequence that is the complement of the first and second known sequences.

2. The method of claim 1 wherein the first and the second known sequences are each 6 nucleotides in length.

3. The method of claim 1 wherein the first and the second known sequences are each 9 nucleotides in length.

4. The method of claim 1 wherein the first and the second known sequences are each 15 nucleotides in length.

5. The method of claim 1 wherein the nucleic acid sample comprises genomic DNA.

6. The method of claim 1 wherein the nucleic acid sample comprises viral genomic nucleic acid.

7. The method of claim 1 wherein the nucleic acid sample is bacterial genomic DNA.

8. The method of claim 1 wherein the nucleic acid sample is mitochondrial DNA.

9. The method of claim 1 wherein the nucleic acid sample is the product of a long range PCR amplification using target specific primers.

10. The method according to claim 1 wherein the second cleavage site is a restriction enzyme recognition site.

11. The method according to claim 1 wherein the first cleavage site comprises a uracil base.

12. The method according to claim 11 wherein the step of cleaving said closed circular probe comprises adding uracil-N-glycosylase to form an abasic site and heating to cleave the at the abasic site.

13. The method according to claim 1 wherein said amplifying is performed by contacting said linear tag probes with at least one universal primer, a polymerase and dNTPs.

14. The method according to claim 2 wherein the first and the second known sequence for each precircle probe combine to form a 12 base sequence, and wherein said plurality of precircle probes comprises more than 1,000,000 different precircle probes each having a different 12 base sequence formed by the first and second known sequences.

15. The method according to claim 14 wherein said oligonucleotide array comprises more than 1,000,000 different tag complements.

16. A method for detecting mutations in a known reference sequence in a nucleic acid sample comprising:
mixing the nucleic acid sample with a plurality of precircle probes under conditions that allow hybridization of precircle probes to complementary target sequences in the nucleic acid sample to form a plurality of hybridization complexes, wherein each precircle probe comprises in the following order:
i. a 5′ target domain complementary to a first sequence of said reference sequence;
ii. a first universal priming site;
iii. a first cleavage site;
iv. a second universal priming site;
v. a tag sequence;
vi. second cleavage site; and
vii. a 3′ target domain complementary to a second sequence of said reference sequence, wherein said first sequence and said second sequence are separated in the reference sequence by a single base, wherein said single base is the interrogation position;
b. extending the 3′ end of said precircle probe by a single base corresponding to the base that is present at that position in the nucleic acid sample;
c. ligating together the ends of precircle probes that are hybridized to target sequences so that the 5′ and 3′ ends of the precircle probe are immediately adjacent to form closed circular probes from a plurality of said precircle probes;
d. optionally digesting any linear probes remaining after step (b);
e. cleaving said closed circular probes at the first cleavage site to obtain linear tag probes;
f. amplifying said linear tag probes to obtain amplified linear tag probes;
g. hybridizing said amplified linear tag probes to an oligonucleotide array comprising probes that are tag complements to obtain a hybridization pattern; and
h. analyzing said hybridization pattern to determine the identity of the base at the interrogation position.

17. The method according to claim 16 wherein step (b) is performed in four separate reactions wherein each reaction contains a different dNTP.

18. The method according to claim 16 wherein step (b) is performed in a single reaction with each dNTP labeled with a distinguishable label.

19. The method according to claim 16 wherein said interrogation position corresponds to a known single nucleotide polymorphism.
